(19) United States
(12) Patent Application Publication
(10) Pub. No.: US 2015/0209465 A1
(43) Pub. Date: Jul. 30, 2015

(54) Volatile Material Dispenser

(71) Applicant: l&l Developments Ltd., Barnet, Hertfordshire (GB)

(72) Inventor: Brian Parry Slade, Ashford (GB)

(73) Assignee: I&I Developments Ltd., Barnet, Hertfordshire (GB)

(21) Appl. No.: 14/422,422

(22) PCT Filed: Aug. 21, 2013
(86) PCT No.: PCT/GB2013/052203

§ 371 Date: Feb. 19, 2015

(30) Foreign Application Priority Data
Aug. 21, 2012 (GB) 1214889.6
Nov. 13, 2012 (GB) 1220407.9

(51) Int. Cl. A61L 9/12

(52) U.S. Cl. CPC A61L 9/12; A61L 9/122

(57) ABSTRACT

An emanator (24, 60) for a volatile material comprises an elongate element having a first plurality of fibres (12, 68) distributed along its length and extending therefrom or substantially perpendicular thereto. The emanator further comprises a fluid pathway for the conveyance of volatile material along the emanator. The pathway comprises one or more second fibres (14, 64) substantially extending in a direction along the length of the element. A system (22, 72) for releasing a volatile material into a room comprises at least one such emanator, a fluid reservoir (26, 74) for a volatile material and a fluid delivery system (28, 76) for delivering fluid from the reservoir to the emanator

This invention relates to dispensing of volatile materials, in particular to an emanator, and a system using the emanator, for dispensing such materials.

Emanators and systems using emanators for dispensing volatile materials, for example room fragrances, insecticides, or the like, are known in the art and come in different forms. This invention relates to a type of emanator supplied from a reservoir of volatile fluid.

Known emanators however have a number of problems associated therewith, in particular known emanators have poor linear release of material, are known to become progressively blocked, the consistency of the emanated product is not maintained over time, and, due to poor emanation, liquid within the reservoir is often discarded at service intervals.

Linear release of material from the emanator is highly desirable as it ensures that a constant performance can be delivered from the emanator. This is beneficial irrespective of the use of the emanator. In, for example, room fragrance delivery it is highly beneficial for the emanator to deliver the same fragrance at substantially the same rate at the end of its life as it is does towards the beginning of its life. When, for example, used for insecticide release it is highly desirable that a linear release of insecticide is achieved over time, thus ensuring effectiveness over the life of the product.

One known design of reservoir which goes some way to addressing some of the known problems associated with emanators is disclosed in International patent application WO 01/77004. This document discloses a pressure compensated dispensing reservoir that assists in delivering linear release of material therefrom by removing or minimising hydrostatic pressure differences over the life of the device by creating and maintaining a substantially constant pressure head in the reservoir which is independent of the height of the liquid therein. However the methods of dispensing the material therefrom do not produce a highly linear output or need interference from an electrically controlled system (for example as disclosed in International patent application WO 01/66158) in order to regulate the dispensing of material therefrom.

The present invention seeks to provide an improved emanator and emanator system.

According to a first aspect of the invention there is provided an emanator for a volatile material, the emanator comprising: an elongate element having a first plurality of fibres distributed along its length and extending substantially perpendicular thereto; and a fluid pathway for the conveyance of volatile material along the emanator, said pathway comprising one or more second fibres substantially extending in a direction along the length of the element.

The first plurality of fibres may comprise a plurality of short fibres attached to a central core. The core may comprise two or more twisted wires and said first plurality of fibres can be retained between said two or more twisted wires.

The one or more second fibres may comprise one or more fibres extending continuously along the core of the element. The second fibres may follow the same twist as the wires. The number of second fibres can be chosen dependent upon whether a low or high output of fragrance is required. For example, the one or more second fibres may comprise one, two three, four, five, six, seven, eight, nine, ten or more fibres, with a low number of fibres being chosen for a low output device, or a higher number being chosen for a greater output.

Optionally the one or more second fibres can be coiled around the core.

In one embodiment of the invention the one or more second fibres deform some of the first fibres such that they extend along the length of the element in an overlapping fashion.

The second fibres can comprise a plurality of strands of polyester or of cotton polyester. The fluid pathway may comprise a capillary pathway. The first plurality of fibres may be polyester.

In a further embodiment the one or more second fibres comprise a subset of said plurality of first fibres, the subset deformed so as to substantially extend in a direction along the length of the element perpendicular to the remainder of the first plurality of fibres.

The emanator may be configured into a shape in which the total length of the flow path of the emanator is greater than the largest dimension of the space envelope occupied by said emanator. The emanator may have a length of between 80 cm and 1.2 m, or between 90 cm and 1.1 m, or be approximately 1 m, and the largest dimension of the space envelope occupied by said emanator may be in the region of 5 to 15 cm, or 7 to 12 cm, or 9 to 11 cm, or approximately 10 cm, for example. The emanator may be configured into one of: a spiral, a conical spiral, a square spiral, a helical coil, and Archimedean spiral.

According to a second aspect of the invention there is provided a system for releasing a volatile material into a room, the system comprising: at least one emanator according to the first aspect of the invention; a fluid reservoir for a volatile material; and a fluid delivery system for delivering fluid from said reservoir to said emanator.

The fluid delivery system may be configured to deliver a volatile fluid to a first end of the at least one emanator, and the at least one emanator may extend downwardly from the first end. Optionally the at least one emanator extends downwardly in a coil around the exterior of the reservoir. Alternatively the fluid delivery system can be configured to deliver a volatile fluid to a first end of the at least one emanator, and the at least one emanator can extend outwardly form the first end in a substantially horizontal plane.

The reservoir can be a pressure compensated reservoir configured to maintain a substantially constant head pressure irrespective of the fluid level within the reservoir.

The emanator may be terminated at one or each end with a sleeve, which may be a thin, and/or permeable sleeve. The sleeve may take the form of a fibrous rod made, and/or may be formed from polyolefine. The sleeve, when placed on an end of the emanator, preferably flattens the first fibres at the emanator end, ensuring the second fibres make good contact with the fluid delivery system.

In an embodiment of the invention the system further comprises a means of creating relative movement between the at least one emanator and the air surrounding it. The means of creating relative movement can comprises a fan configured to move air past the at least one emanator. Optionally the fan may be configured to draw air in an upwards direction over past the at least one emanator. Alternatively the means of creating relative movement may comprise means, for example a motor and gears, for rotating the at least one emanator. In a further alternative arrangement the means of creating relative movement may comprise a means for repeatedly imparting a magnetic field upon the emanator to cause it to oscillate. The means of imparting a magnetic field may comprise an electromagnetic coil and a controller to drive said coil so as to provide a pulsed magnetic field acting on said emanator.

In the system of the invention the at least one emanator may further comprise an absorbent mass at the distal end thereof. Preferably the absorbent material is a pad and, more preferably, the absorbent material is a cellulose pad. In an embodiment, the cellulose pad may have a thickness of approximately 3 mm, although pads of other thicknesses, e.g. between 2.5 mm and 3.5 mm may also be utilized. The pad is preferably positioned so that no part of it is below the constant level reservoir. The pad may be oriented in a horizontal plane to avoid creating a hydrostatic head. In another arrangement the absorbent mass may comprise a continuous plurality of sections of the elongate element arranged such that perpendicularly extending first fibres of one section intermesh with the perpendicularly extending first fibres of at least one other adjacent section. In an alternative arrangement the absorbent mass may comprise an absorbent material adjacent the end of the at least one emanator and in contact therewith.

In an embodiment of the invention the fluid delivery means can further comprise a mechanical diverter to divert a flow path for volatile material from the reservoir to none, one, or more than one, of the at least one emanators.

An enclosure may be provided as part of the system for enclosing at least the reservoir and at least one emanator, the enclosure being provided with vents to allow the flow of air into and out of the enclosure.

According to a third aspect of the invention there is provided a system for releasing a volatile material into a room, the system comprising at least one emanator comprising: an elongate element having a first plurality of fibres distributed along its length and extending therefrom; and a fluid pathway for the conveyance of volatile material along the emanator, said pathway comprising one or more second fibres substantially extending in a direction along the length of the element; a fluid reservoir for a volatile material; and a fluid delivery system for delivering fluid from said reservoir to said emanator.

In embodiments, the first plurality of fibres comprises a plurality of short fibres attached to a support which runs along the length of the emanator. The one or more second fibres may comprise one or more fibres extending continuously along the support. The support may be polyester. The second fibres may be attached to the surface or at least partially embedded of the polymer material, and said first fibres may extend from the surface of the polymer layer on which the second fibres are attached.

It will be appreciated that any of the optional features of the third aspect of the invention may be used in the system of the second aspect of the invention, which differs in that the design of the emanator itself is different.

According to a fourth aspect of the invention there is provided a method of manufacturing an emanator according to the first aspect of the invention, the method comprising: providing two or more elongate wires; providing a first plurality of short fibres arranged to pass between at least two of the two or more elongate wires; providing one or more second fibres aligned with the two or more wires; and twisting the wires and second fibres to trap the first fibres therebetween so that they extend substantially perpendicularly thereto.

According to a fifth aspect of the invention there is provided a method of manufacturing an emanator of the invention, the method comprising: providing an emanator precursor comprising an elongate element having a first plurality of short fine fibres attached to a core and extending substantially perpendicular thereto; and wrapping one or more second fibres tightly in a helical pattern along the core.

The method may further comprise deforming at least some of the first plurality of fibres with the one or more second fibres, so that the deformed first fibres extend substantially along the direction of the core.

In an embodiment, the method of the fourth or fifth aspect may further comprise delivering the emanator, via a secondary rotatable member, to a primary rotatable member at an acute angle with respect to the axis of rotation thereof, the diameter of the primary rotatable member being greater than that of the secondary rotatable member. The primary rotatable member, which may be a cylinder or mandrel, may be the master or driver component, caused to rotate by a driving means e.g. a lathe. The secondary rotatable member, which may also be a cylinder or mandrel, may be a slave component that is free to rotate. The emanator may be delivered to the secondary cylinder under high tension. Preferably, the primary and secondary cylinders are positioned in close proximity to each other and such that their centres of rotation are aligned.

The method of either the fourth or fifth aspect of the invention may further comprise forming the emanator into one of: a spiral, a conical spiral, a square spiral, a helical coil, and Archimedean spiral. The spiral, conical spiral, square spiral, helical coil, or Archimedean spiral may be formed by altering the tension and/or said angle during the winding of the emanator onto said primary member.

The above described method is not constrained to forming emanators and so, according to a sixth aspect of the invention, a method of winding a metal strip or wire onto a primary rotatable member comprises delivering a metal strip or wire, via a secondary rotatable member, to a primary rotatable member at an acute angle with respect to the axis of rotation thereof, the diameter of the primary rotatable member being greater than that of the secondary rotatable member. The primary rotatable member, which may be a cylinder or mandrel, may be the master or driver component, caused to rotate by a driving means e.g. a lathe. The secondary rotatable member, which may also be a cylinder or mandrel, may be a slave component that is free to rotate. The wire or strip may be delivered to the secondary cylinder under high tension. Preferably, the primary and secondary cylinders are positioned in close proximity to each other and with their centres of rotation are aligned. The wire or strip may be formed into one of a spiral, conical spiral, square spiral, helical coil, or Archimedean spiral by altering the tension and/or said angle during the winding of the wire or strip onto said primary member.

Embodiments of the invention are described below, by way of example, with reference to the accompanying drawings in which:

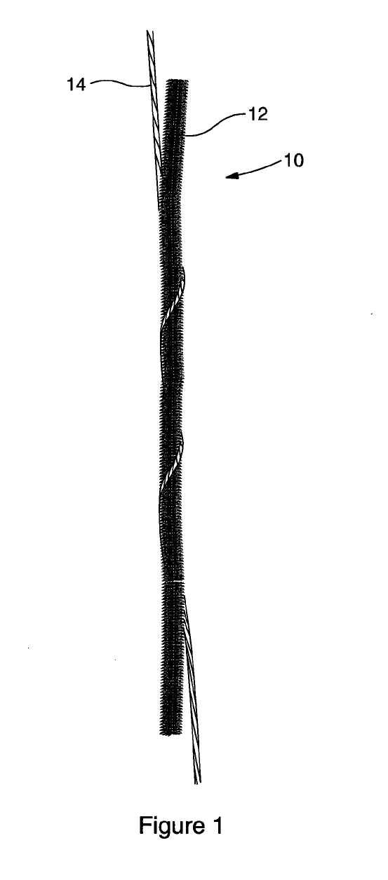

FIG. 1 shows an emanator element precursor;

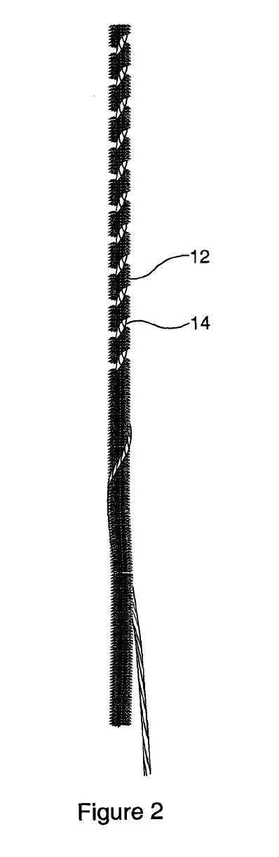

FIG. 2 shows an emanator element of the present invention part way through construction:

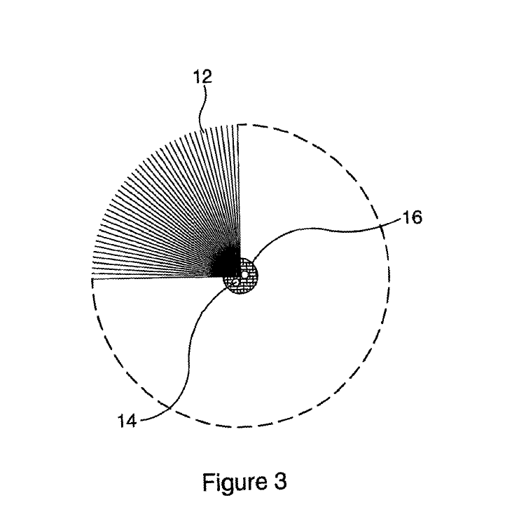

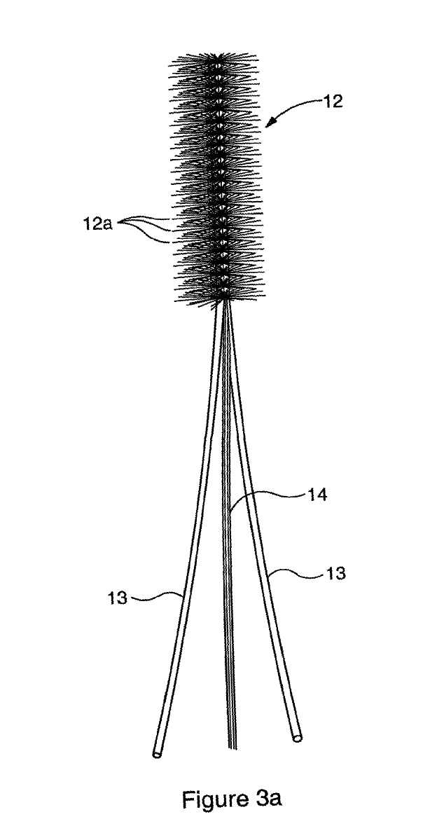

FIG. 3 shows a cross section through an emanator element of the present invention;

FIG. 3a shows an emanator element of another embodiment of the present invention part way through construction;

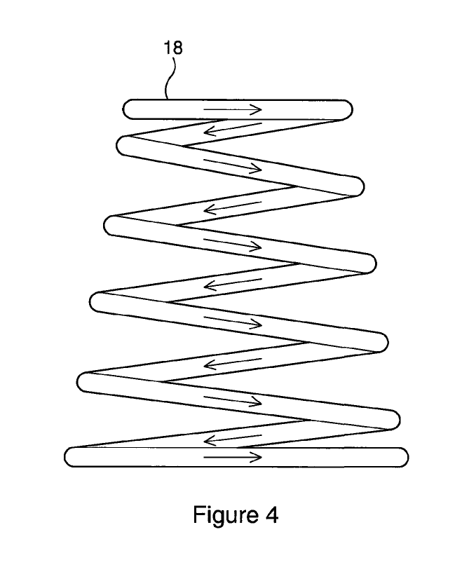

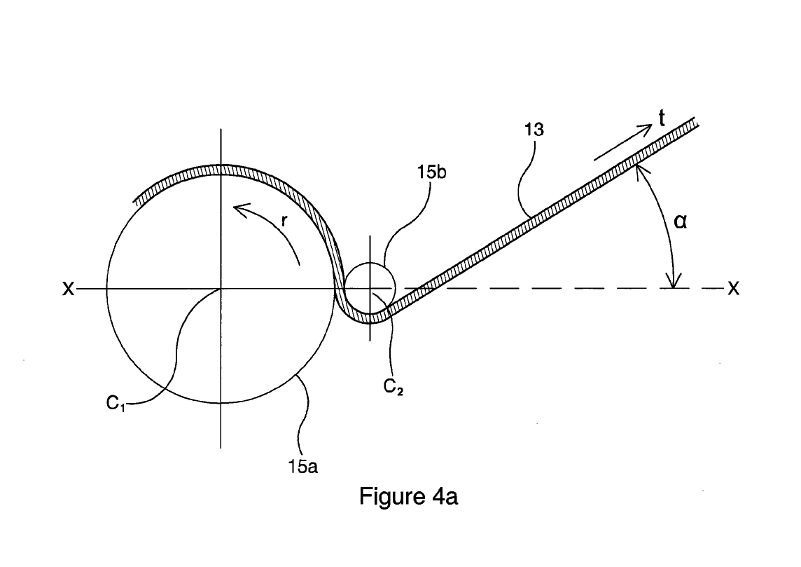

FIG. 4 shows one embodiment of the emanator element of the invention;

FIG. 4a illustrates a method for forming a spiral emanator according to embodiments of the invention;

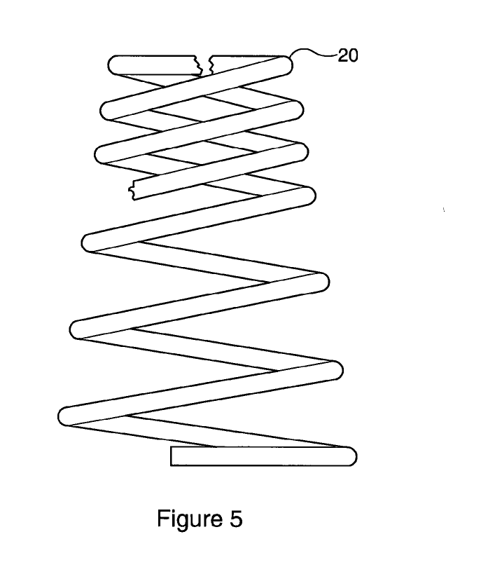

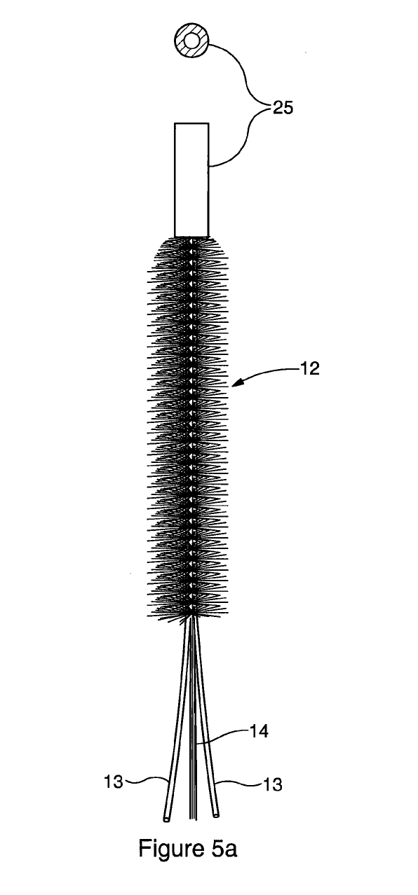

FIG. 5 shows another embodiment of the emanator element of the invention;

FIG. 5a shows the emanator of the embodiment of FIG. 3a with a sleeve over one end thereof, together with an end view of the sleeve;

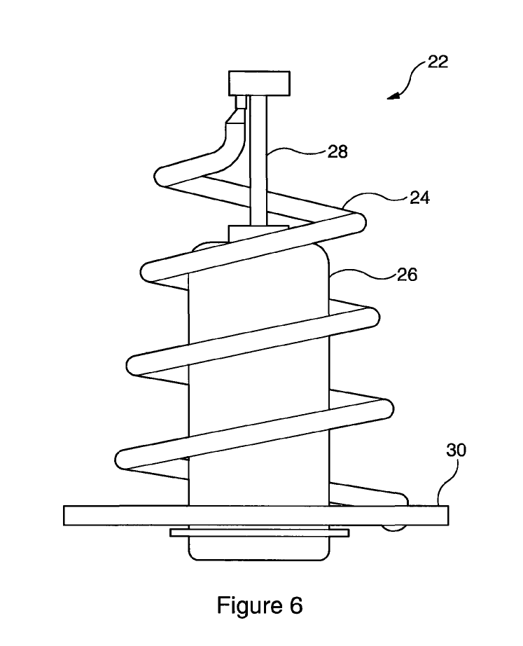

FIG. 6 shows a system of one embodiment of the invention;

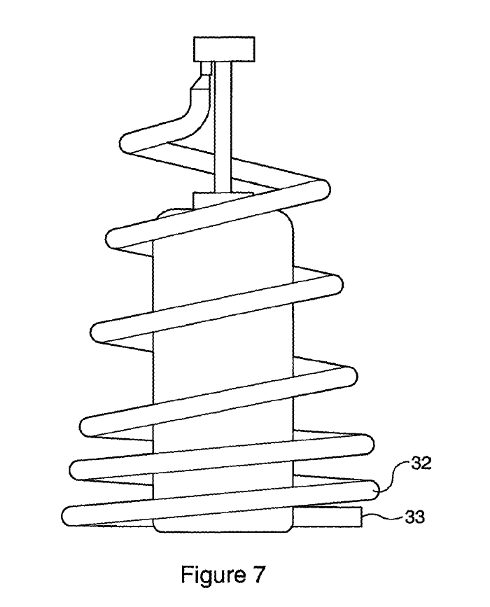

FIG. 7 shows a system of a further embodiment of the invention;

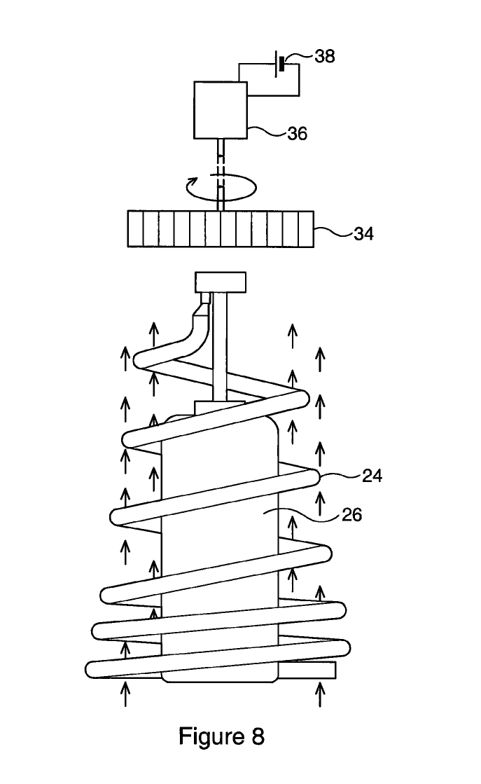

FIG. 8 shows a system of the invention with a fan;

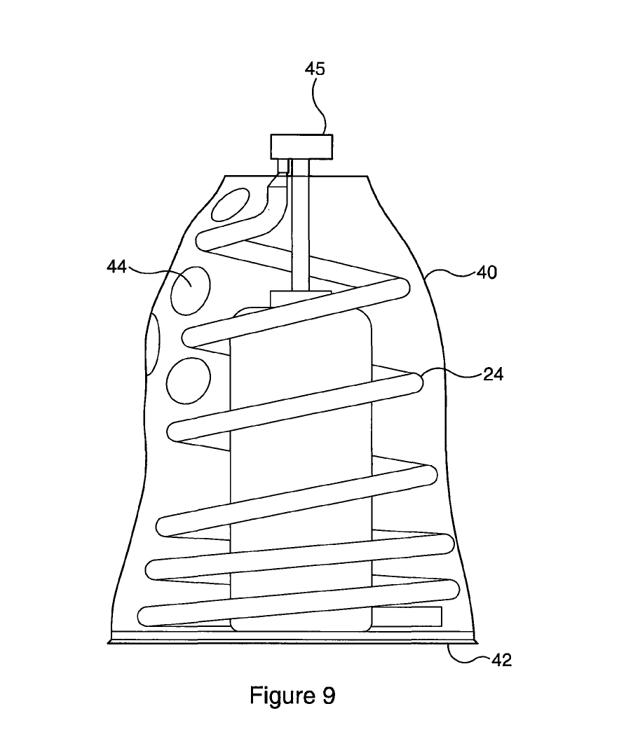

FIG. 9 shows a system of another embodiment of the invention;

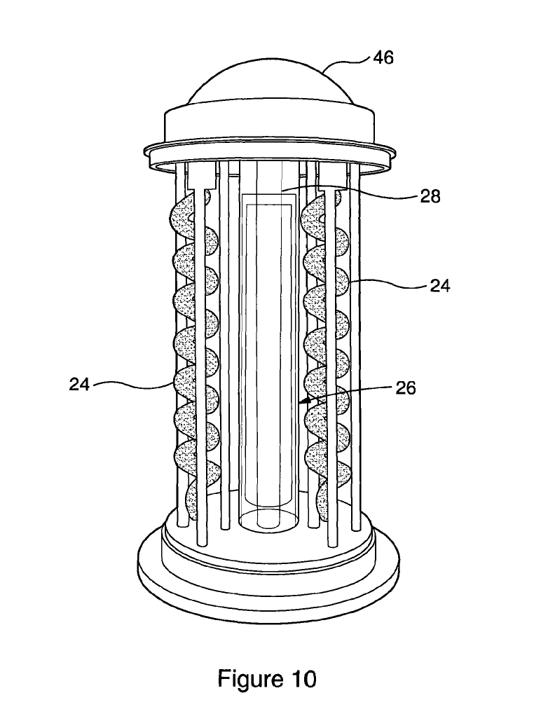

FIG. 10 shows a system of an embodiment of the invention with off-set emanators;

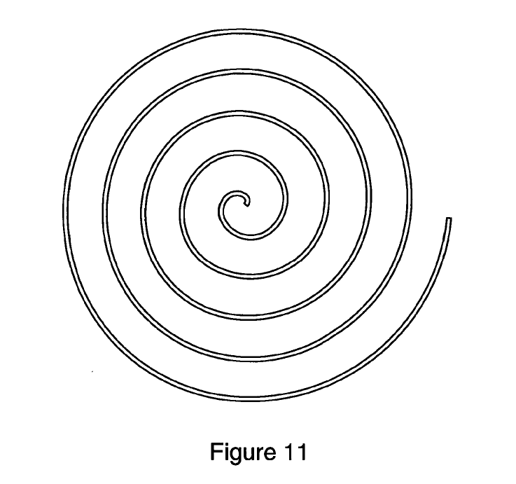

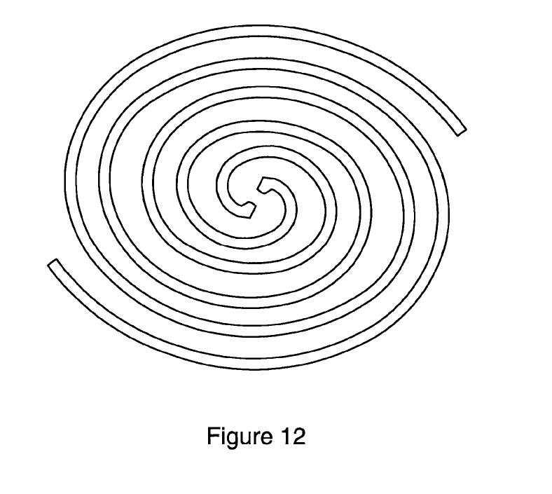

FIGS. 11 and 12 show single and double flat spiral shaped emanators of the invention;

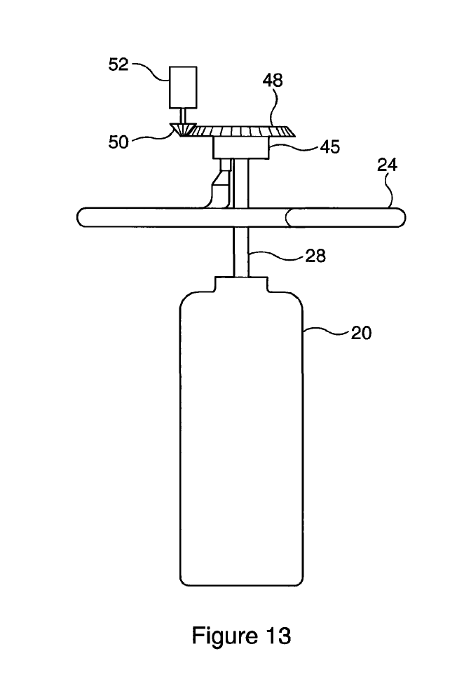

FIG. 13 shows a system of the invention having a flat spiral shaped emanator;

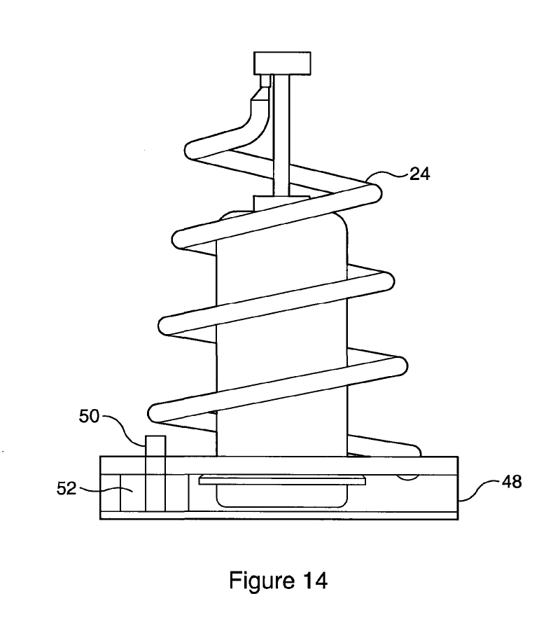

FIG. 14 shows a system of the invention in which the emanator is moved by an electromagnetic field;

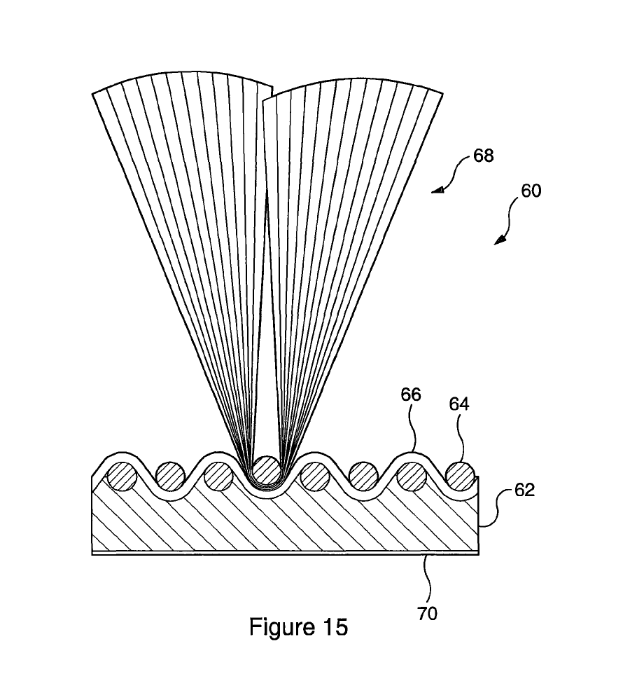

FIG. 15 shows a cross section of an emanator used in the system of the third aspect of the invention;

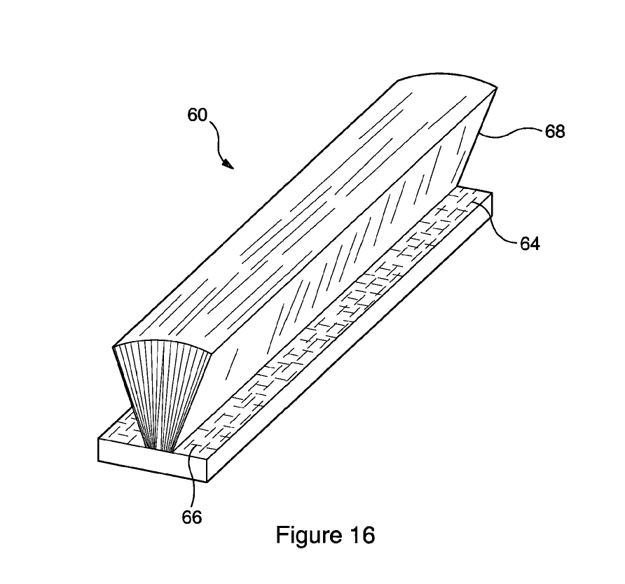

FIG. 16 shows a perspective view of a section of the emanator used in the system of the third aspect of the invention;

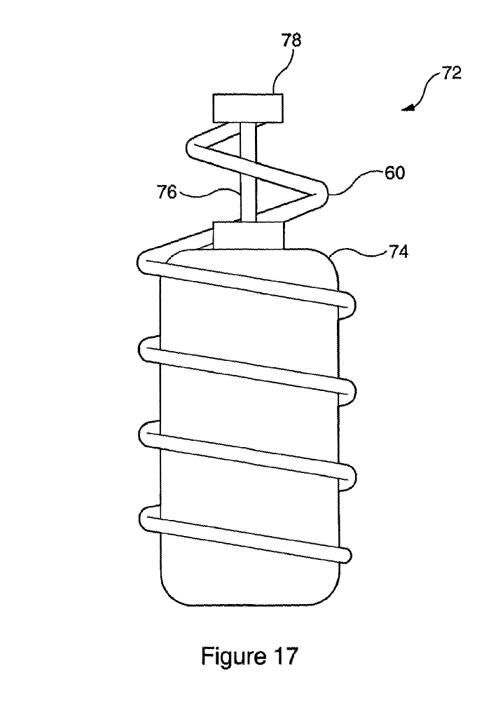

FIG. 17 shows a system according to one embodiment of the third aspect of the invention;

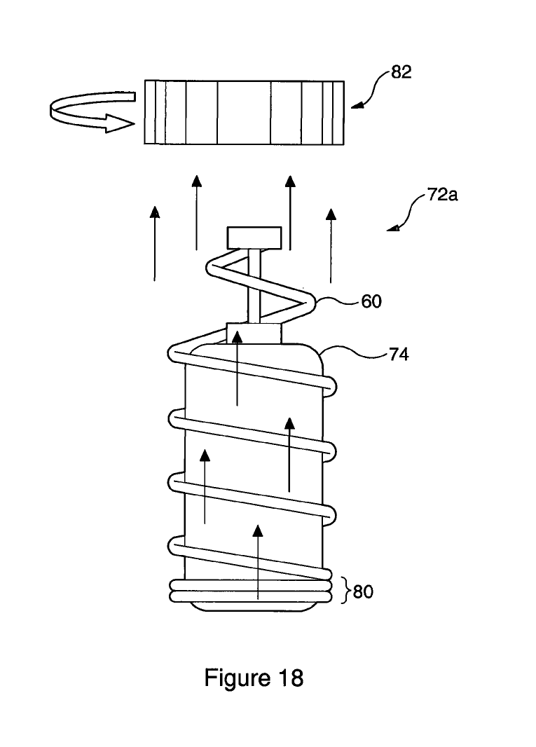

FIG. 18 shows a system according to an alternative embodiment of the third aspect of the invention; and

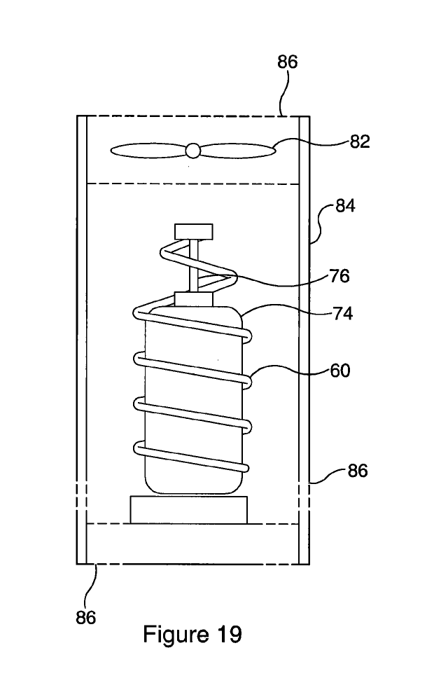

FIG. 19 shows a system according to a further embodiment of the third aspect of the invention contained within an enclosure.

Referring to FIG. 1 a precursor of one embodiment of the emanator of the invention is shown. The precursor comprises an elongate element 10 having a first plurality of short fine polyester fibres 12 that extend substantially perpendicular thereto, and a number of fibres of cotton polyester thread 14. The polyester threads are retained on the element by virtue of being trapped between two twisted pieces of wire, which form a self-supporting longitudinal axis, i.e. the construction is very similar to that of a craft item commonly referred to as a pipe cleaner. The self-supporting nature of the two twisted wires (emanator support wires) allows air to freely circulate around the fibres 12. The emanator support wires are conveniently made from stainless steel, as this prevents oxidization that could otherwise occur as a result reaction with components of the formulation, although it will be appreciated that other materials could also be utilized. Conveniently, the support wires are approximately 0.4 mm in diameter, although wires of other diameters may also be used.

Referring to FIGS. 2 and 3, to construct the emanator of the invention second fibres, which may be in the form of threads 14, are wrapped tightly around the element 10. As part of the wrapping process some of the first plurality of fibres 12 become trapped underneath the threads 14 and are deformed to form, together with the threads 14, a second group of fibres 16 that lie substantially along the length of the element 10. The threads 14 and the deformed fibres form a flow path for the transport of the volatile material along the length of the emanator and enable a gradual flow of the volatile material along the element and outwardly into the fibres 12. Fluid flow along the emanator may be due to capillary action, or may be by means of other fluid transfer mechanisms, including gravity induced flow.

An alternative way of making the emanator of the invention, which can result in a larger emanating surface area, is to lay second fibres in the form of the longitudinal threads 14 alongside the wires of the emanator prior to the wires being twisted to trap the fibres. In this way the threads 14 are maintained centrally in the emanator without the reduction in the number of short fibres 12. The threads 14 are twisted together with the wires and form a central flow path, which as discussed above may be a capillary flow path, along the axis of the emanator. The lower half of FIG. 3a shows the emanator support wires 13 and the longitudinal threads 14 prior to twisting together. The upper half of FIG. 3a shows the constructed emanator.

As this method results in the flow path formed by the second fibres 14 being totally surrounded by the first plurality of fibres this will reduce direct evaporation from the thread 14 which will result in an ever greater improvement of fluid flow along the emanator. The number of threads (or “core yarns”) 14 provided can be varied to provide high or low output devices as required. For example, one or two core yarns 14 could be utilised for a low output device; three, four, five, six, seven, eight, nine, ten or more threads 14 could be utilized where the output is required to be greater.

Twisting the support wires physically separates the first (emanating) fibres 12 into ‘tufts’ 12a. The core yarns 14 are in contact with the radial emanating fibres 12 as a result of being trapped between the wires during construction. This provides a continuous capillary circuit from one end of the emanator to the other A pathway is created for a fluid to travel along by being influenced by the forces of gravity to act on separate opposing columns of the circuit to control the supply of liquid to the emanator.

The spacing created between adjacent tufts of the radial emanating fibres 12 is particularly useful in providing an emanating system that is highly permeable to air, which is useful for efficiently evaporate the fluid, and the density of the emanating fibres 12 is thus chosen accordingly to maximise the amount of exposed surfaces for efficient evaporation of the liquid product. I.e. it is desirable not to pack the fibres 12 too densely as this would decrease the emanator's permeability to air.

The emanator may be formed into any convenient shape which maximises the length of the emanator whilst minimising its volume, or space envelope. Referring to FIG. 4 the emanator is shown formed into a conical spiral. The emanator has an inlet end which is supplied with the volatile material. The volatile material flows along the length of the emanator by travelling along the flow path formed by threads 14. As it flows along the emanator the fluid also flows out along the fibres 12 which, due to their fine nature have a large surface area to allow for evaporation. Generally the volatile material will be a mixture of different materials, all of which evaporate at a slightly different rate. The materials which evaporate fastest will evaporate from the upper end of the coil and the materials which evaporate more slowly will travel further down the emanator prior to moving into the fibres 12 and evaporating therefrom. As the emanator has a conical spiral shape the length of each coil increases from the top of the coil towards the bottom. Therefore, the slower evaporating components of the volatile material, which flow along the central flow path formed by the threads to the lower coils, actually have a larger surface area from which to evaporate as each turn of the spiral in that part of the emanator is longer. The emanating surface area of the spiral emanator is directly proportional to its length. The flow rate can be controlled by altering the characteristics of the spiral e.g. by increasing or decreasing the helix angle and/or length.

Embodiments of the invention thus provide constant delivery of fluid through the provision of a multi-coiled emanator having a long path length to provide enough time for evaporation. The spiral path provides continuous ‘irrigation’ to maintain high performance of the product.

In the emanator there are three system effects that interact with one another, those being the rate of flow of the material, the rate of evaporation of the material and the temperature. For an efficient system there needs to be a balance of these factors that can cope with temperature fluctuations, and the shape of the coil can assist in providing a stable system, that is one with reliable and repeatable evaporation characteristics. Any section of the length of the coil represents a certain surface area available for evaporation. The liquid flows down the coil from the top to the bottom and at lower temperatures the liquid product travels further down the spiral and is therefore evaporated over a longer length of the emanator. This provides a degree of temperature compensation by making a larger surface area available at lower temperatures to enable a more consistent evaporation rate therefrom. Conversely, at higher temperatures the fluid may fully evaporate before it reaches the end of the emanator.

As discussed above, an emanator in the form of a conical spiral has several advantages. A method of forming such an emanator will now be described, with reference to FIG. 4a. Generally speaking, a large, primary cylinder or mandrel 15a is driven e.g. by a lathe (not shown) such that it is caused to rotate in the direction r as indicated by the arrow (anti-clockwise in FIG. 5a). A length of emanator, comprising the two twisted support wires 13, the first fibres 12 and the second fibres 14, is delivered to the primary cylinder 15a in order to create the desired spiral shape. However, in order to create and maintain the desired tightness of the spiral, the emanator is delivered to the primary cylinder via a secondary cylinder 15b utilizing a ‘crowbar’ or ‘lever’ effect as will be described below.

The secondary cylinder 15b is of much smaller diameter, and is free to rotate about its axis (indicated by the crosshairs at c₂). The emanator is delivered to the forming system 15a, 15b under high tension (i.e. the tension force acts in a direction t away from the forming system 15a, 15b). The primary and secondary cylinders 15a, 15b are positioned in close proximity to each other, i.e. with only a small gap approximately the size of the emanator therebetween, and such that their centres of rotation, c₁ and c₂ respectively, are aligned with an axis X-X (shown horizontally in FIG. 5a). The emanator is delivered to the secondary cylinder 15b and then to the primary cylinder 15a at an acute angle α with respect to the centres of rotation c₁, c₂ of the cylinders and to the horizontal axis X-X in FIG. 5a.

The secondary cylinder 15b, having a diameter much less than that of the primary cylinder 15a, produces a small diameter curve for the emanator being fed to the primary cylinder 15a. This small curve is opposite to the curve being formed on the large cylinder and, as such, has a greater structural strength that the larger curve of the primary cylinder and therefore exerts a greater force on the wires 13 of the emanator.

During the winding of the emanator on to the primary cylinder 15a, the coils thus produced can be wound onto the cylinder 15a so as to be spaced from each other along the length of the cylinder 15a, in order to produce a spiral of the desired length. This can be achieved by drawing the emanator along the length of the cylinders 15a, 15b during the winding process, and the cylinders 15a, 15b are of an appropriate length to accommodate the spiral windings.

In one embodiment, a larger cylinder 15a having a diameter of 30 mm and a smaller cylinder 15b having a diameter of 8 mm produced a spiral having a mean diameter of approximately 50 mm. It will, however, be appreciated that cylinders of other sizes e.g. a large cylinder having a diameter in the range substantially between 20-40 mm, or 25-35 mm, and a small cylinder having a diameter substantially in the range 4-12 mm, or 6-10 mm, could be used. In this embodiment, the delivery angle α was approximately 35°. Again, it will be appreciated that the angle can be altered to suit requirements, e.g. with the angle being substantially between 25 and 40° with respect to the horizontal. In any event, the resultant diameter produced is always greater than the diameter of the cylinder on which it is wound.

For a given tension, the size of the spiral being formed by this method is dependent upon the angle α. For example, the greater the angle α to the horizontal, the smaller the resultant mean diameter of the spiral produced; conversely, the smaller the angle α, the greater the mean diameter of the resultant coil. Therefore, a conical coil can be produced by winding the emanator onto the primary cylinder 15a, progressively changing the angle α between the start and finish thereof. Alternatively, the angle α can be kept constant, and the applied tension varied to produce a similar effect: a higher tension would result in a smaller mean diameter of the coils, and vice versa.

Providing the wires 13 of the emanator at the acute angle α (relative to the axis of the cylinders 15a 15b) to a smaller cylinder prior to winding around a larger cylinder has been found to be a convenient way to form the desired conical spiral shape for the emanator, and especially when using stainless steel wires 13. Using stainless steel advantageously avoids the problem of oxidization/corrosion mentioned above, although it was found to be difficult to form stainless steel into the desired configuration using conventional methods.

The method described with reference to FIG. 4a is equally applicable to forming wires and strips of metal, rather than emanators specifically.

FIG. 5 shows another emanator design in which a second coil 20 (partially cut away) is provided intertwined with the coil of FIG. 4, thereby doubling the effective evaporation surface area. The coils may be connected in parallel or in series. If they are connected in series, one coil, at its top, is connected to the supply of volatile fluid and the coils are either connected together at the bottom or are a continuation of each other. This series combination can replace the need for a sink by providing an additional length of the emanator for evaporation and doubling the path length whilst at the same time maintaining the input end of the pathway at the highest point.

In the embodiments discussed above, and as shown ion FIG. 5a, the emanator 24 may be terminated, at one or each end, with a thin, permeable sleeve 25. The sleeve 25 may take the form of a fibrous rod made, for example, of polyolefine. The sleeve 25, when placed on the end of the emanator 24, flattens the radial fibres 12 at the emanator end, ensuring the transport fibres 14 make good contact with the fluid delivery system 28, described below.

Referring to FIG. 6 a system 22 for releasing a volatile material into a room is shown. The system 22 comprises an emanator 24 as described above which is connected to a fluid reservoir 26 by a fluid delivery system 28. The reservoir 26 can be used to supply a constant flow of a liquid fragrance to an emanating element 24 from which the fragrance is dispersed into the surrounding environment.

The reservoir 26 and fluid delivery system 28 are preferably the same as those disclosed in WO01/77004 the teachings of which are incorporated herein by reference, and in particular that described in relation to FIGS. 6 to 12 of that document, except in that the emanator 24 of the present invention, as described herein above, is used as the evaporative element of the system. Embodiments of the present invention thus provide separate zones for transmitting and emanating the fluid, which leads to improved clarity of fragrance since all of the ‘notes’ of the fragrance can be emanated together.

The reservoir has a pressure regulating means in it that ensures that the pressure at the bottom of a wick element of the fluid delivery system, that delivers fluid from the bottom of the container to the start of the emanator, is maintained substantially constant independently of the height of fluid in the container. This is achieved by means of the upper end of the container being sealed and an air inlet into the container being provided adjacent or at the same level as the outlet. In this manner a negative pressure develops above the fluid in the upper sealed end of the container that balances and acts against the pressure created by the fluid head height above the air inlet. Essentially the fluid pressure at the outlet is therefore maintained substantially at atmospheric pressure. Further details about the system are found in the above mentioned patent.

The system 22 of the invention can also include an absorbent element 30 which acts as a sink at the lower end of the emanator. As the fluid passes down the emanators known in the art, if the components of the fluid flowing along the emanator, in particular those components having a slower evaporation rate, reach the end of the emanator without evaporating then prior art emanators can become blocked with these slower evaporating fluids. These start to back up in the emanator, in particular along the flow path transporting the volatile material, gradually reducing the length of emanator to which the flow path is capable of delivering new fluid. This can, over time, change the composition of the material being evaporated from the emanator resulting in unacceptable eminence quality. By providing this “sink” at the end of the emanator any excess material can be allowed to flow from the end of the emanator, thereby preventing the flow paths along the emanator becoming backed up. The rate at which fluid enters the sink is much less than the rate at which the sink can evaporate fluid. This enables the sink to collect and retain any residues and solids that would normally block the capillaries, whilst emanating the remaining fluid, so that all of the product can be released linearly over time.

NB. This is a dynamic fluid system of emanating fragrance material, with a constant flow of fluid entering the top of the emanator and exiting from the bottom of the emanator after evaporating most of the product. The small amount of fluid exiting the bottom of the emanator into the sink 30 is important for the irrigation of the fluid circuit to maintain a high performance.

It has been found that providing a sink in the form of a porous sheet material provides the necessary absorbency. The sink 30 shown in the example of FIG. 6 is a cellulose pad. A cellulose pad having a thickness of approximately 3 mm has been found to have the absorbent qualities desired, although it will be appreciated that pads 3 of other thicknesses, e.g. between 2.5 mm and 3.5 mm also work well. In any event, the sink 30 is preferably positioned so that no part of it is below the constant level reservoir. The sink 30 may be oriented in a horizontal plane to avoid creating a hydrostatic head.

Referring to FIG. 7 another system of the invention is shown in which the emanator coils 32, 33 are placed closer together at the lower end of the spiral, preferably in an overlapping fashion. In this manner the lower turns of the coil essentially act as the absorbent element providing the sink.

Referring to FIG. 8 a system of the invention is shown which, in addition to the system of FIG. 7, also comprises a fan disposed above the reservoir 26 and emanator 24. The fan 34 is coupled to an electric motor 36 which is powered by a battery 38. Alternatively, the fan may be powered by a supply of mains electricity. The fan rotates in a direction to draw a flow of air over the coil in an upwards direction as depicted by the arrows. The system may be contained within an enclosure (not shown) having ventilation openings therein to allow for the flow of air therethrough. The enclosure, fan and motor may form a re-usable part of the system and the reservoir, delivery system and emanator may form a disposable part of the system that can be changed on a regular basis.

Referring to FIG. 9 a further embodiment of the invention is shown in which the reservoir 26 and emanator 24 are covered by a shroud 40 that connects to a base 42 to substantially enclose the system. The shroud 40 is provided with a plurality of ventilation openings 44 which cover its surface, although as will be appreciated only a few are shown for illustrative purposes. The ventilation openings 44 allow a flow of air in and around the emanator 24. The top 45 of the fluid delivery system 28 extends above the shroud 40 so that it is accessible to the user. As described in WO 01/77004, pressing down on the top of the fluid delivery system can be a means of breaking a frangible seal between the system and the fluid within the reservoir to allow the system to commence dispense of the volatile material.

FIG. 10 depicts an alternative embodiment of a system of the invention. This system comprises a central reservoir 26 which is pressure compensated, and a fluid delivery system, again as described in WO 01/77004. In this embodiment the system is provided with three emanators 24 (one hidden from view), each of which is connected to the top of the fluid delivery system 28 to receive fluid at their upper ends from the reservoir 24. A selector cap 46 is provided that is rotatable to direct the flow of volatile material from the fluid delivery system 28 to none, one, two or all of the emanators 24. In this way a variable dispense can be achieved that can be stopped or started at a user's discretion. A ventilated shroud (not shown) may optionally surround the system below the cap 46 so as to prevent inadvertent contact with the wetted emanators 24 during use. Although depicted with three spiral shaped emanators it will be appreciated that this design could include two or more emanators.

Although the above embodiments all disclose emanators which have a vertical component to fluid flow therein, it is also anticipated that the emanators of the present invention could have only a horizontal flow path. For example the emanators could be as shown in FIG. 11 (single spiral), or FIG. 12 (double spiral). In these embodiments the emanator can be used as a static emanator. The emanator may be used with the same reservoir and fluid delivery system as described hereinabove.

A further embodiment of the system of the invention is shown in FIG. 13. This embodiment has a reservoir 26 and fluid delivery system 28 as described above, and a flat spiral shaped emanator 24 as shown in FIG. 11, although it will be appreciated that the emanator of FIG. 12 could also be used. In this embodiment the top 45 of the fluid delivery system 28 is provided with a bevel gear 48 which is driven by a second bevel gear 50 connected to a motor 52 so to rotate the emanator 24. This increases evaporation due to the relative motion of emanator and air, and also can assist in the flow of material along the emanator by centrifugal force. The example of a drive mechanism using bevel gears 50, 52 is shown for illustrative purposes and it will be appreciated by the skilled person that any drive means for rotating the emanator 24 may be used, and that the emanator, although depicted as a flat spiral, could be replaced with an emanator in the form of a conical spiral or helix. The direction of rotation of the emanator should be such as to influence the flow of fluid in a direction towards the end of the emanator.

Referring to FIG. 14 yet another embodiment a system of the invention is shown. In this embodiment a base 48 is provided below the emanator 24. The base contains a ferrous core 50 and a coil 52 surrounding at least a portion of the core 50. The coil 52 is connected to a source of electricity, which may for example be a battery or a mains supply, via an electronic circuit that is configured to intermittently supply electricity to the coil 52. This creates an electromagnetic field that magnetises the core 50 so as to create an attractive magnetic force on the emanator, which has a centre made of ferrous wire. In the preferred embodiment where the emanator support wires 13 are made of stainless steel, which is non-magnetic, a small ferrous plate (not shown) is attached to the emanator. As only a short pulse of electricity is provided the emanator experiences a short electromagnetic force on it which is then released. As the emanator 24 is formed into a coil, when it is attracted and released it will continue to oscillate as, due to its coiled nature it displays some spring like qualities.

This oscillation induced by the electromagnetic effect of the coil 52 creates relative motion between the emanator 24 and the air surrounding it thereby promoting evaporation therefrom. The pulsed electromagnetic field created in the coil 52 and core 50 requires only a minimal amount of energy.

It will be appreciated by the skilled person that although described as being in the base 48, providing the electromagnetic field produced acts on the emanator 24, the positioning of the coil 52 and core 50 are only dictated by packaging requirements and may be placed in any suitable position.

Referring to FIG. 15 a cross section through an emanator 60 used in the system of the invention is shown. The emanator comprises a polymer support 62 into which a length of woven fabric of longitudinal 64 and traverse 66 polyester threads is partially embedded. Although shown as single threads for simplicity, each traverse 66 and longitudinal 64 thread comprises a plurality of fine fibres lying adjacent each other.

Within the fabric is a plurality of tufts of polyester fibres 68 that are trapped in the fabric such that loose ends thereof extend substantially perpendicular to the support 62. As can be seen the fibres 68 are only retained at their lower end and are loose at their upper end and fan out slightly as they extend away from the support 62. In use the longitudinal polyester threads 64 act as a capillary fluid pathway along which the volatile material can travel along the emanator, and the fibres 68 extending from the support give a large surface area from which the volatile material can evaporate. The traverse threads 66 act to transport the volatile material from the outer longitudinal threads to the fibres 68.

The polymer support 62 is made of a heat softenable polymer and the polymer is softened and the woven fabric pressed into its surface so as to attach thereto. In this manner the top surface of the fabric is exposed. Although the threads 64, 66 could transport the volatile material using capillary action if they were completely encapsulated, having them exposed on the surface assists in the transfer from the woven fabric to the fibres 68 and also assists in the emanator drawing the volatile material into it as this may occur on the exposed top side of the fabric which presents a larger surface area than would be exposed by the end of the threads 64, 66 of the fabric were fully encapsulated.

The emanator may be provided with a self-adhesive backing strip 70 by which it can be conveniently attached to a surface if required. It will be appreciated that this feature is optional and it is not a requirement of the invention that the emanator is adhered to a surface.

Referring to FIG. 16 a section of the emanator 60 is shown. As can be seen the threads 64 and 66 of the woven fabric extend along the length of the emanator and the longitudinal threads 64 they form a continuous capillary fluid pathway along the length of the emanator. As can also be seen the fibres 68 also extend along the length of the emanator 60 so as to form a strip pile.

Referring to FIG. 17 a system 72 of the invention is shown. The system releases a volatile material into a room. The system 72 comprises an emanator 60 as described above which is formed into a coil shape and which is connected to a fluid reservoir 74 by a fluid delivery system 76.

The reservoir 74 and fluid delivery system 76 are preferably the same as those disclosed in WO01/77004 the teachings of which are incorporated herein by reference, and in particular that described in relation to FIGS. 6 to 12 of that document, except in that the emanator 60 of the present invention, as described herein above, is used as the evaporative element of the system. The reservoir has a pressure regulating means in it that ensures that the pressure at the bottom of a wick element of the fluid delivery system, that delivers fluid from the bottom of the container to the start of the emanator, is maintained substantially constant independently of the height of fluid in the container. This is achieved by means of the upper end of the container being sealed and an air inlet into the container being provided adjacent or at the same level as the outlet. In this manner a negative pressure develops above the fluid in the upper sealed end of the container that balances and acts against the pressure created by the fluid head height above the air inlet. Essentially the fluid pressure at the outlet is therefore maintained substantially at atmospheric pressure. Further details about the system are found in the above mentioned patent.

The emanator coils from the top 78 of the fluid delivery system down around the exterior of the reservoir 74. The emanator 60 may be pre formed into the desired coil shape by heating it to soften the polymer support, forming it into the required shape, and then cooling the polymer support so as to maintain its required shape. The emanator 60 may be attached only to the top 78 of the fluid delivery system 76 and be freely suspended therefrom, or, alternatively the emanator 60 may be adhered to the outside of the reservoir 74, for example by use of the self-adhesive strip 70. As will be appreciated, if the emanator 60 is adhered to the exterior of the reservoir 74 it would not be necessary to pre-form it into a coil shape prior to adhesion. As the fibres 68 of the present invention only extend in one direction from the support 62, and as when winding the spiral for the emanator the fibres will extend outwardly from the fabric, forming the emanator into a coil opens out the fibres of the tufts away from one another thereby exposing a large emanating surface area and at the same time increasing its permeability to air. This greatly improves the evaporation of volatile material from the emanator. Due to the improved ability of this design of emanator to evaporate volatile material, it is anticipated that the emanator length using this design can be reduced compared to the emanator design of FIG. 2, resulting in a more compact system.

The top 78 of the fluid delivery system has a slot (not shown) formed therein into which the end of the emanator can be inserted such that fluid from the reservoir can contact the longitudinal fibres running along the emanator 60 so as to be transported therealong.

Referring now to FIG. 18 a system 72A is shown having some of these additional features. A number of turns of the emanator 60 are coiled around the reservoir 74 at its lower end so as to form an absorbent element 80 which acts as a sink at the lower end of the emanator. As the fluid passes down the emanators known in the art, if the components of the fluid flowing along the emanator, in particular those components having a slower evaporation rate, reach the end of the emanator without evaporating then prior art emanators can become blocked with these slower evaporating fluids. These start to back up in the emanator, in particular along the flow path transporting the volatile material, gradually reducing the length of emanator to which the flow path is capable of delivering new fluid. This can, over time, change the composition of the material being evaporated from the emanator resulting in unacceptable eminence quality. By providing this “sink” at the end of the emanator any excess material can be allowed to flow from the end of the emanator, thereby preventing the flow paths along the emanator becoming backed up.

In addition the system 72A also comprises a fan 82 disposed above the reservoir 74 and emanator 60. The fan 82 is coupled to an electric motor (not shown) which is powered by a source of electricity, for example a battery or a supply of mains electricity. The fan rotates in a direction to draw a flow of air over the coil of emanator 60 in an upwards direction as depicted by the arrows.

As shown in FIG. 19, the system may be contained within an enclosure 84 having ventilation openings 86 therein to allow for the flow of air therethrough. The enclosure 84, fan 82 and motor (omitted for clarity) may form a re-usable part of the system and the reservoir 74, delivery system 76 and emanator 60 may form a disposable part of the system that can be changed on a regular basis. As will be appreciated by the skilled person the enclosure 84 is also suitable for containing the system shown in FIGS. 1 to 14, and as described above in relation thereto.

It will be appreciated that the system described herein with reference to FIGS. 15 to 19 is a further development of the system described in relation to FIGS. 1 to 14. Accordingly the skilled person will understand that the emanator described in relation to the former may be used in the embodiments illustrated for the latter, or vice versa, including, without limitation: the sink of FIG. 6, the fan and motor system of FIG. 8, the shroud of FIG. 9 or the rotating mechanism of FIG. 13 thereof. Furthermore, although described as having one emanator, it will be appreciated that the system described with respect to FIGS. 15 to 19 may comprise two emanators arranged as shown in FIG. 5.

### Test Results

Tests were run on an emanator system of the invention. A reservoir, emanator and an evaporative sink as shown in FIG. 6 were produced.

To make the emanator two craft pipe cleaners were joined together and placed in tension in a lathe and two lengths of polyester cotton were then tightly wound around the pipe cleaner in a helical fashion by rotating the pipe cleaner with the lathe and gradually moving the polyester cotton along its length as it rotated. Once made the emanator was formed into a helical shape and attached to a pressure compensated reservoir as detailed hereinabove. The reservoir contained Hoshi Hula 463182B fragrance produced by Firmenich. Although not necessary for the functioning of the emanator, it may, in some circumstances, be beneficial to clean the emanator element prior to use, preferably as part of the manufacturing process. Cleaning may, for example, remove any residue or coatings on the core of the emanator that could otherwise become dissolved in the volatile material as it passes along the emanator. Although it is not believed that this would affect the evaporation of fluid from the emanator, it may, for example, lead to discolouration which could provide an undesirable visual effect.

To test the invention the emanator, reservoir and evaporative sink were housed in a ventilated enclosure containing a fan. The fan was electronically controlled through a cyclic pattern of on/off with an off time of ninety seconds followed by an on time of sixty seconds.

Daily measurements of the mass of the system were taken and the weight loss, in grams per twenty four hour period, was calculated over a 22 day period. The system was run continuously day and night for the duration of the test and the temperature was uncontrolled and therefore variable. The weight was measured at substantially the same time each day by placing the system on a Satorious Universal Electronic Scale, and the readings recorded. Weight loss per 24 hour period was then calculated.

As can be seen although there is some day to day variation in the weight loss there is no overall trend and the weight loss due to emanation is substantially linear over time. The fluctuations, such as they exist, may be influenced by factors such as differing temperatures on a day to day basis.

| June | g/24 h |
|---|---|
| 19 | 0.33 |
| 20 | 0.32 |
| 21 | 0.34 |
| 22 | 0.28 |
| 23 | 0.30 |
| 24 | 0.27 |
| 25 | 0.31 |
| 26 | 0.36 |
| 27 | 0.37 |
| 28 | 0.39 |
| 29 | 0.40 |
| 30 | 0.33 |

| July | g/24 h |
|---|---|
| 1 | 0.30 |
| 2 | 0.27 |
| 3 | 0.27 |
| 4 | 0.28 |
| 5 | 0.32 |
| 6 | 0.34 |
| 7 | 0.30 |
| 8 | 0.26 |
| 9 | 0.27 |
| 10 | 0.26 |
| 11 | 0.23 |
| 12 | 0.26 |
| 13 | 0.27 |
| 14 | 0.28 |

The test was also repeated without utilizing a fan, and the daily measurements for a period of 28 days are shown below.

| | g/24 h |
|---|---|
| August | |
| 31 | 0.21 |
| September | |
| 1 | 0.24 |
| 2 | 0.30 |
| 3 | 0.26 |
| 4 | 0.36 |
| 5 | 0.40 |
| 6 | 0.36 |
| 7 | 0.33 |
| 8 | 0.37 |
| 9 | 6.38 |
| 10 | 0.37 |
| 11 | 0.41 |
| 12 | 0.36 |
| 13 | 0.26 |

| July | g/24 h |
|---|---|
| 14 | 0.27 |
| 15 | 0.36 |
| 16 | 0.33 |
| 17 | 0.32 |
| 18 | 0.32 |
| 19 | 0.28 |
| 20 | 0.27 |
| 21 | 0.26 |
| 22 | 0.25 |
| 23 | 0.25 |
| 24 | 6.23 |
| 25 | 0.34 |
| 26 | 0.39 |
| 27 | 0.37 |

Again, although there is some day to day variation in the weight loss, and the weight loss due to emanation is substantially linear over time. The fluctuations, such as they exist, may be influenced by factors such as differing temperatures on a day to day basis. The average daily weight loss over this 28 day period is 0.31 g/24 hours, and the total fragrance released was 8.8 g.

It will be appreciated by the skilled person that the various features of the different embodiments described above may be used in combination with the features of different embodiments.

1.-65. (canceled)

66. An emanator for a volatile material, the emanator comprising: an elongate element having a first plurality of fibres distributed along its length and extending substantially perpendicular thereto; and a fluid pathway for the conveyance of volatile material along the emanator, said pathway comprising one or more second fibres substantially extending in a direction along the length of the element.

67. An emanator according to claim 66 wherein said first plurality of fibres comprises a plurality of short fibres attached to a central core.

68. An emanator according to claim 67 wherein the one or more second fibres comprise one or more fibres extending continuously along the core of the element.

69. An emanator according to claim 68 wherein said core comprises two or more twisted wires and said first plurality of fibres are retained between said two or more twisted wires.

70. An emanator according to claim 69 wherein said second fibres follow the same twist as said wires.

71. An emanator according to claim 68 wherein said one or more second fibres are coiled around said core.

72. An emanator according to claim 71 wherein the one or more second fibres deform some of said first fibres so that they extend along the length of the element in an overlapping fashion.

73. An emanator according to claim 67 wherein said one or more second fibres comprises a subset of said plurality of first fibres, the subset deformed so as to substantially extend in a direction along the length of the element perpendicular to the remainder of said first plurality of fibres.

74. An emanator according to claim 66 configured into into a shape in which the total length of the flow path of the emanator is greater than the largest dimension of the space envelope occupied by said emanator, into one of: a spiral, a conical spiral, a square spiral, a helical coil, and Archimedean spiral.

75. An emanator according to claim 66 in which said fluid pathway comprises a capillary pathway.

76. A system for releasing a volatile material into a room, the system comprising:
at least one emanator according to claim 66;
a fluid reservoir for a volatile material; and
a fluid delivery system for delivering fluid from said reservoir to said emanator.

77. The system according to claim 76 wherein said fluid delivery system is configured to deliver a volatile fluid to a first end of said at least one emanator, and said at least one emanator extends downwardly from said first end.

78. The system according to claim 77 wherein said at least one emanator extends downwardly in a coil offset from the reservoir.

79. The system according to claim 76 wherein said fluid delivery system is configured to deliver a volatile fluid to a first end of said at least one emanator, and said at least one emanator extends outwardly from said first end in a substantially horizontal plane.

80. A system for releasing a volatile material into a room, the system comprising:
at least one emanator comprising: an elongate element having a first plurality of fibres distributed along its length and extending therefrom; and a fluid pathway for the conveyance of volatile material along the emanator, said pathway comprising one or more second fibres substantially extending in a direction along the length of the element;
a fluid reservoir for a volatile material; and
a fluid delivery system for delivering fluid from said reservoir to said emanator.

81. The system according to claim 80 wherein said first plurality of fibres comprises a plurality of short fibres attached to a support which runs along the length of the emanator.

82. The system according to claim 81 wherein the one or more second fibres comprise one or more fibres extending continuously along the support.

83. The system according to claim 80 wherein the support comprises a polymer material and the second fibres are attached to the surface or at least partially embedded of the polymer material, and said first fibres extend from the surface of the polymer layer on which the second fibres are attached.

84. The system according to claim 83 comprising a woven fibre mat attached to said polymer layer and wherein said second fibres comprise longitudinal fibres of said woven fibre mat, and wherein said first fibres extend from said woven fibre mat.

85. The system according to claim 80 wherein the first fibres comprise a plurality of adjacent tufts of fibres extending along the length of the emanator, said fibres within each tuft extending in a divergent manner so as to form a substantially continuous pile.

86. The system according to claim 80 wherein the emanator is configured into a shape in which the total length of the flow path of the emanator is greater than the largest dimension of the space envelope occupied by said emanator, into in the form of one of a spiral, a conical spiral, a helical coil, or an Archimedean spiral.

87. The system according to claim 80 in which said fluid pathway comprises a capillary pathway.

88. The system according to claim 80 wherein said fluid delivery system is configured to deliver a volatile fluid to a first end of said at least one emanator, and said at least one emanator extends downwardly from said first end.

89. The system according to claim 80 wherein an end of said emanator extends into an upper end of the fluid delivery system.

90. The system according to claim 77 wherein said at least one emanator extends downwardly in a coil around the exterior of said reservoir.

91. The system according to claim 76 wherein said reservoir is a pressure compensated reservoir configured to maintain a substantially constant head pressure irrespective of the fluid level within said reservoir.

92. The system according to claim 80 further comprising a means of creating relative movement between the at least one emanator and the air surrounding it.

93. The system according to claim 76 wherein the at least one emanator further comprises an absorbent mass at the distal end thereof.

94. The system according to claim 93 wherein said absorbent mass comprises a continuous plurality of sections of said elongate element arranged such that perpendicularly extending first fibres of one section intermesh with the perpendicularly extending first fibres of at least one other adjacent section.

95. The system according to claim 76 wherein said fluid delivery means further comprises a mechanical diverter to divert a flow path for volatile material from the reservoir to none, one, or more than one, of said at least one emanator.

96. The system according to claim 76 further comprising an enclosure for enclosing at least said reservoir and at least one emanator, said enclosure being provided with vents to allow the flow of air into and out of said enclosure.

97. The system according to claim 92 wherein the means of creating relative movement comprises a means for repeatedly imparting a magnetic field upon the emanator to cause it to oscillate.

98. A method of manufacturing an emanator according to claim 66, said method comprising:
providing two or more elongate wires;
providing a first plurality of short fibres arranged to pass between at least two of said two or more elongate wires;
providing one or more second fibres aligned with said two or more wires; and
twisting said wires and second fibres to trap said first fibres therebetween so that they extend substantially perpendicularly thereto.

99. A method of manufacturing an emanator according to claim 66, said method comprising:
providing an emanator precursor comprising an elongate element having a first plurality of short fine fibres attached to a core and extending substantially perpendicular thereto; and
wrapping one or more second fibres tightly in a helical pattern along said core.

100. The method of claim 99 further comprising
deforming at least some of said first plurality of fibres with the one or more second fibres, so that said deformed first fibres extend substantially along the direction of said core.

101. The method of claim 98, further comprising delivering the emanator, via a secondary rotatable member, to a primary rotatable member at an acute angle with respect to the axis of rotation thereof, the diameter of the primary rotatable member being greater than that of the secondary rotatable member.

102. The method of claim 101, further comprising delivering the emanator to the secondary rotatable member under high tension.

103. The method of claim 101, wherein the primary and secondary rotatable members are positioned in close proximity to each other and such that their centres of rotation are aligned.

104. The method according to claim 98 comprising forming said emanator into one of: a spiral, a conical spiral, a square spiral, a helical coil, and Archimedean spiral, which is formed by altering the tension and/or said angle during the winding of the emanator onto said primary member.
